(19) United States
(12) Patent Application Publication
(10) Pub. No.: US 2006/0040917 A1
(43) Pub. Date: Feb. 23, 2006

(54) Benzoxazepine derivatives as selective estrogen receptor modulators

(76) Inventors: James C. Lanter, Raritan, NJ (US); Zhihua Sui, Raritan, NJ (US); James J. Fiordeliso, Raritan, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/191,499

(22) Filed: Jul. 28, 2005

Related U.S. Application Data
(60) Provisional application No. 60/602,054, filed on Aug. 17, 2004.

(51) Int. Cl. A61K 31/553; C07D 267/02

(52) U.S. Cl. 514/211.09; 540/546

(57) ABSTRACT

The present invention is directed to novel benzoxazepine derivatives, pharmaceutical compositions containing them and their use in the treatment of disorders and diseases mediated by an estrogen receptor.

### FIELD OF THE INVENTION

The present invention is directed to novel benzoxazepine derivatives, pharmaceutical compositions containing them and their use in the treatment or prevention of disorders and diseases mediated by an estrogen receptor such as hot flashes, vaginal dryness, osteopenia, osteoporosis, hyperlipidemia, loss of cognitive function, degenerative brain diseases, cardiovascular, cerebrovascular diseases, hormone sensitive cancers and hyperplasia (in tissues including breast, endometrium, and cervix in women and prostate in men), endometriosis, uterine fibroids, osteoarthritis; and as contraceptive agents either alone or in combination with a progestogen or progestogen antagonist. The compounds of the invention are selective estrogen receptor modulators.

### BACKGROUND OF THE INVENTION

Estrogens are a group of female hormones essential for the reproductive process and for the development of the uterus, breasts, and other physical changes associated with puberty. Estrogens have an effect on various tissues throughout a woman's body, not only those involved in the reproductive process, such as the uterus, breasts, and external genitalia, but also tissues in the central nervous system, bones, the liver, skin, and the urinary tract. The ovaries produce most of the estrogens in women's body.

Menopause is defined as the permanent cessation of menses due to loss of ovarian follicular function and the almost termination of estrogen production. The midlife transition of menopause is characterized by a decrease in estrogen that provokes both short-term and long-term symptoms with the vasomotor, urogenital, cardiovascular, skeletal and centra nervous systems, such as hot flushes, urogenital atrophy, increased risk of cardiovascular disease, osteoporosis, cognitive and psychological impairment, including an increased risk of cognitive disorders and Alzheimer's disease (AD).

Seventy-five percent of all women experience some occurrence of vasomotor symptoms associated with the onset of menopause such as body sweating and hot flushes. These complaints may begin several years before menopause and in some women may continue for more than 10 years either relatively constant, or as instant attacks without a definable, provoking cause.

Urogenital symptoms associated with the onset of menopause involving the vagina include a sensation of dryness, burning, itching, pain during intercourse, superficial bleeding and discharge, along with atrophy, stenosis. Symptoms involving the urinary tract include a burning sensation during urination, frequent urgency, recurrent urinary tract infections, and urinary incontinence. These symptoms have been reported to occur in up to 50% of all women near the time of menopause and are more frequent a few years after menopause. If left un-treated, the problems can become permanent.

Heart attack and stroke are major causes of morbility and mortality among senior women. Female morbility from these diseases increases rapidly after menopause. Women who undergo premature menopause are at greater coronary risk than menstruating women of similar age. The presence of serum estrogen has a positive effect on serum lipids. The hormone promotes vasodilation of blood vessels, and enhances the formation of new blood vessels. Thus the decrease in serum estrogen levels in postmenopausal women results in adverse cardiovascular effect. Additionally, it is theorized that differences in the ability of blood to coagulate may account for the observed difference in the occurrence of heart disease before and after menopause.

The skeleton is under a continuous process of bone degeneration and regeneration in a carefully regulated interaction among the bone cells. These cells are directly affected by estrogen. Estrogen deficiency results in a loss of bone structure, and decrease of bone strength. Rapid loss of bone mass during the year immediately following menopause leads postmenopausal osteoporosis and increased risk of fracture.

Estrogen deficiency is also one of the causes for the degenerative changes in the central nervous system and may lead to Alzheimer's disease and decline of cognition. Recent evidence suggests an association between estrogen, menopause, and cognition. More particularly, it has been reported that estrogen replacement therapy and the use of estrogen in women may prevent the development of AD, and improve cognitive function.

Hormone replacement therapy (HRT)—more specifically estrogen replacement therapy (ERT)—is commonly prescribed to address the medical problems associated with menopause, and also to help hinder osteoporosis and primary cardiovascular complications (such as coronary artery disease) in both a preventive and therapeutical manner. As such, HRT is considered a medical therapy for prolonging the average life span of postmenopausal women and providing a better quality of life.

ERT effectively relieves the climacteric symptoms and urogenital symptoms and has shown significant benefits in the prevention and treatment of heart disease in postmenopausal women. Clinical reports have shown that ERT lowered heart attack rates and mortality rates in populations that received ERT versus similar populations not on ERT. ERT initiated soon after menopause may also help maintain bone mass for several years. Controlled investigations have shown that treatment with ERT has a positive effect even in older women up to age of 75 years.

However, there are numerous undesirable effects associated with ERT that reduce patient compliance. Venous thromboembolism, gallbladder disease, resumption of menses, mastodynia, and a possible increased risk of developing uterine and/or breast cancer are the risks associated with ERT. Up to 30% of women who were prescribed with ERT do not fill the prescription, and the discontinuation rate is between 38% and 70%, with safety concerns, and adverse effects (bloating and break-through bleeding) the most important reasons for discontinuation.

A new class of pharmacological agents known as Selective Estrogen Receptor Modulators or SERMs have been designed and developed as alternatives for HRT. Raloxifene, a nonsteroidal benzothiophere SERM is marketed in the US and Europe for the prevention and treatment of osteoporosis under the trademark of Evista®. Raloxifene has been shown to reduce bone loss and prevent fracture without adversely stimulating endometrial and mammary tissue, though raloxifene is somewhat less efficacious than ERT for protecting against bone loss. Raloxifene is unique and differs significantly from ERT in that it does not stimulate the endometrium and has the potential for preventing breast cancer. Raloxifene has also demonstrated beneficial estrogen agonist effects on cardiovascular risk factors, more specifically through a rapid and sustained decrease in total and low-density lipoprotein cholesterol levels in patients treated with raloxifene. In addition, raloxifene has been shown to reduce plasma concentration of homocysteine, an independent risk factor for atherosclerosis and thromboembolic disease.

However, raloxifene has been reported to exacerbate symptoms associated with menopause such as hot flushes and vaginal dryness, and does not improve cognitive function in senior patients. Patients taking raloxifene have reported higher rates of hot flashes compared with either placebo or ERT users and more leg cramps than placebo users, although women who took ERT had a higher incidence of vaginal bleeding and breast discomfort than raloxifene or placebo users.

As yet, neither raloxifene nor any of the other currently available SERM compounds has been shown to have the ability to provide all the benefits of currently available ERT such as controlling postmenopausal syndrome and preventing AD, without causing adverse side effects such as increasing risk of endometrial and breast cancer and bleeding. Thus there exists a need for compounds which are selective estrogen receptor modulators and which provide all of the benefits of ERT while also addressing the vasomotor, urogenital and cognitive disorders or conditions associated with the decrease in systemic estrogen associated with menopause.

### SUMMARY OF THE INVENTION

The present invention is directed to a compound of formula (I) 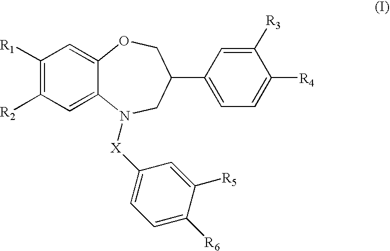
wherein:
R₁, R₂, R₃, and R₄ are selected form the group consisting of hydrogen, halogen, hydroxy, alkyl, alkoxy, acyloxy, silyloxy and hydroxy substituted lower alkyl, provided that at least one of R₁, R₂, R₃ or R₄ is hydroxy, alkoxy, silyloxy or acyloxy; X is selected from the group consisting of CH₂, CO or SO₂;
R5 and R6 are each independently selected from the group consisting of hydrogen, halogen, alkoxy, lower alkyl, —O—(CH₂)₂₋₃—Cl, —O—(CH₂)₂₋₃—OH, and —O—(CH₂)₂₋₃—NR^{A}R_{B}, provided that if present only one of R5 or R6 is —O—(CH₂)₂₋₃—NR^{A}R^{B};
wherein, R^{A} and R^{B} are each independently selected from the group consisting of hydrogen and lower alkyl; alternatively R^{A} and R^{B} are taken together with the N atom to which they are bound to form a five to six membered heteroaryl or a five to six membered heterocycloalkyl group; or a pharmaceutically acceptable salt thereof.

In a preferred embodiment of the invention, one of R₁ and R₂ is selected from the group consisting of hydroxy, alkoxy, silyloxy or acyloxy and one of R₃ and R₄ is selected from the group consisting of hydroxy, alkoxy, silyloxy and acyloxy.

In another preferred embodiment of the invention, R^{A} and R^{B} are independently selected from the group consisting of hydrogen methyl and ethyl or are taken together with the nitrogen atom to which they are bound to form pyrrolodinyl, morpholinyl or piperidinyl.

In one aspect, the invention relates to a pharmaceutical composition comprising a pharmaceutically acceptable carrier and any of the compounds described above. In another aspect, the invention relates to a pharmaceutical composition made by mixing any of the compounds described above and a pharmaceutically acceptable carrier. In yet another aspect, the invention is a process for making a pharmaceutical composition comprising mixing any of the compounds described above and a pharmaceutically acceptable carrier.

The invention also provides methods of treating a disorder mediated by one or more estrogen receptors in a subject in need thereof comprising administering to the subject a therapeutically effective amount of any of the compounds or pharmaceutical compositions described above.

In a preferred embodimet, the invention provides a method of contraception comprising administering to a subject in need thereof co-therapy with a therapeutically effective amount of a compound of formula (I) with a progestogen or progestogen antagonist.

In yet another aspect, the invention relates to the use of any of the compounds described herein in the preparation of a medicament for treating: (a) hot flashes, (b) vaginal dryness, (c) osteopenia, (d) osteoporosis, (e) hyperlipidemia, (f) loss of cognitive function, (g) a degenerative brain disorder, (h) cardiovascular disease, (i) cerebrovascular disease (j) breast cancer, (k) endometrial cancer, (I) cervical cancer, (m) prostate cancer, (n) benign prostatic hyperplasia, (o) endometriosis, (p) uterine fibroids, (q) osteoarthritis and for (r) contraception in a subject in need thereof.

### DETAILED DESCRIPTION OF THE INVENTION

The present invention is directed to compounds of formula (I), 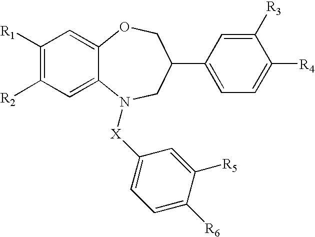
wherein R¹, R², R³, R⁴, R⁵, R⁶ and X are as defined above. Where the compounds according to this invention have at least one chiral center, they may accordingly exist as enantiomers. The compounds of the present invention are modulators of an estrogen receptor, useful for the treatment and prevention of disorders associated with estrogen depletion, including, but not limited to hot flashes, vaginal dryness, osteopenia, osteoporosis, hyperlipidemia, loss of cognitive function, degenerative brain diseases, cardiovascular and cerebrovascular diseases); for the treatment of hormone sensitive cancers and hyperplasia (in tissues including breast, endometrium, and cervix in women and prostate in men); for the treatment and prevention of endometriosis, uterine fibroids, and osteoarthritis; and as contraceptive agents either alone or in combination with a progestogen or progestogen antagonist.

Representative compounds of the present invention are as listed in Table 1. The abbreviations “OMOM” and “OPiv” refer to methoxymethoxy and pivaloyl groups, respectively, attached to the aryl ring via the oxygen. The abbreviations “NPi” and “NMo” refer to piperidine and morpholine groups, respectively, attached to the alkyl chain via the nitrogen.

TABLE 1

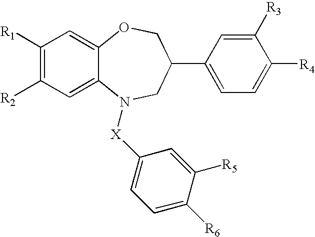

| ID# | X | R₁ | R₂ | R₃ | R₄ | R₅ | R₆ | MS |
|---|---|---|---|---|---|---|---|---|
| 1 | CH₂ | OTBS | H | OH | H | H | O(CH₂)₂Npi | 589^{a} |
| 2 | CH₂ | OTBS | H | OH | H | H | O(CH₂)₂Cl | 538^{b} |
| 3 | CH₂ | OTBS | H | H | OTBS | H | O(CH₂)₂Cl | 654^{a} |
| 4 | CH₂ | OH | H | H | OH | H | O(CH₂)₂Cl | 424^{b} |
| 5 | CO | OMe | H | OMe | H | H | O(CH₂)₂Npi | 517^{a} |
| 6 | CO | OH | H | OH | H | H | O(CH₂)₂Npi | 489^{a} |
| 7 | CH₂ | OH | H | OH | H | H | O(CH₂)₂Npi | 475^{a} |
| 8 | CO | OMe | H | OMe | H | H | O(CH₂)₂NMe2 | 477^{a} |
| 9 | CO | OH | H | OH | H | H | O(CH₂)₂NMe2 | 449^{a} |
| 10 | CO | OMe | H | H | OMe | H | O(CH₂)₂Npi | 517^{a} |
| 11 | CO | OMe | H | OMe | H | H | O(CH₂)₂OH | 450^{a} |
| 12 | CH₂ | OH | H | H | OH | H | O(CH₂)₂Npi | 475^{a} |
| 13 | CO | OH | H | OH | H | O(CH₂)₂NPi | H | 489^{a} |
| 14 | CO | OH | H | OH | H | H | O(CH₂)₂Cl | 438^{b} |
| 15 | CO | OAc | H | OAc | H | H | O(CH₂)₂Cl | 524^{a} |
| 16 | CH₂ | OAc | H | OAc | H | H | O(CH₂)₂Npi | 559^{a} |
| 17 | CO | OH | H | H | OH | H | H | 362^{b} |
| 18 | CH₂ | OH | H | H | OH | O(CH₂)₂Cl | H | 424^{b} |
| 19 | CH₂ | OMOM | H | H | OMOM | O(CH₂)₂Cl | H | 514^{a} |
| 20 | CH₂ | OMOM | H | H | OMOM | O(CH₂)₂NPi | H | 563^{a} |
| 21 | CH₂ | OMOM | H | H | OMOM | O(CH₂)₂NMo | H | 565^{a} |
| 22 | CH₂ | OH | H | H | OH | O(CH₂)₂NPi | H | 475^{a} |
| 23 | CH₂ | OH | H | H | OH | O(CH₂)₂NMo | H | 477^{a} |
| 24 | CH₂ | OH | H | OH | H | O(CH₂)₂Cl | H | 424^{b} |
| 25 | CH₂ | OH | H | OH | H | O(CH₂)₂NPi | H | 475^{a} |
| 26 | CH₂ | OPiv | H | H | OPiv | O(CH₂)₂NPi | H | 643^{a} |
| 27 | CH₂ | OH | H | OMe | H | O(CH₂)₂NPi | H | 489^{a} |

Note:
^{a}MH+;
^{b}M − H

As used herein, the term “alkyl” whether used alone or as part of a substituent group, include straight and branched chains, preferably, a chain containing one to eight carbon atoms. For example, alkyl radicals include, but are not limited to methyl, ethyl, propyl, isopropyl, butyl, isobutyl, sec-butyl, t-butyl, pentyl and the like. Unless otherwise noted, “lower” when used with alkyl means a carbon chain composition of 1-4 carbon atoms.

As used herein, unless otherwise noted, “alkoxy” shall denote an oxygen ether radical of the above described straight or branched chain alkyl groups. For example, methoxy, ethoxy, n-propoxy, sec-butoxy, t-butoxy, n-hexyloxy and the like. Unless otherwise noted, “lower” when used with alkoxy means an alkoxy group comprising 1-4 carbon atoms. Alkoxy also includes methoxymethoxy.

As used herein, unless otherwise noted, “acyloxy” shall denote an carbonyl oxy radical of the above described straight or branched chain alkyl groups. For example, acetoxy, propionyloxy, pivalyloxy, and the like. Unless otherwise noted, “lower” when used with acyloxy means an acyloxy group comprising 1-4 carbon atoms.

As used herein, unless otherwise noted, “silyloxy” shall denote an silyl oxy radical of the above described straight or branched chain alkyl groups. For example, trimethylsilyloxy, triethylsilyloxy, t-butylsilyloxy and the like. Unless otherwise noted, “lower” when used with alkoxy means an alkoxy group comprising 1-4 carbon atoms.

As used herein, unless otherwise noted, the term “cycloalkyl” shall mean any stable 3-8 membered monocyclic, saturated ring system, for example cyclopropyl, cyclobutyl, cyclopentyl, cyclohexyl, cycloheptyl and cyclooctyl.

As used herein, unless otherwise noted, “heteroaryl” shall denote any five or six membered monocyclic aromatic ring structure containing at least one heteroatom selected from the group consisting of O, N and S, optionally containing one to three additional heteroatoms independently selected from the group consisting of O, N and S; or a nine or ten membered bicyclic aromatic ring structure containing at least one heteroatom selected from the group consisting of O, N and S, optionally containing one to four additional heteroatoms independently selected from the group consisting of O, N and S. The heteroaryl group may be attached at any heteroatom or carbon atom of the ring such that the result is a stable structure.

Examples of suitable heteroaryl groups include, but are not limited to, pyrrolyl, furyl, thienyl, oxazolyl, imidazolyl, purazolyl, isoxazolyl, isothiazolyl, triazolyl, thiadiazolyl, pyridyl, pyridazinyl, pyrimidinyl, pyrazinyl, pyranyl, furazanyl, indolizinyl, indolyl, isoindolinyl, indazolyl, benzofuryl, benzothienyl, benzimidazolyl, benzthiazolyl, purinyl, quinolizinyl, quinolinyl, isoquinolinyl, isothiazolyl, cinnolinyl, phthalazinyl, quinazolinyl, quinoxalinyl, naphthyridinyl, pteridinyl, and the like.

As used herein, the term “heterocycloalkyl” shall denote any five to seven membered monocyclic, saturated or partially unsaturated ring structure containing at least one heteroatom selected from the group consisting of O, N and S, optionally containing one to three additional heteroatoms independently selected from the group consisting of O, N and S; or a nine to ten membered saturated, partially unsaturated or partially aromatic bicyclic ring system containing at least one heteroatom selected from the group consisting of O, N and S, optionally containing one to four additional heteroatoms independently selected from the group consisting of O, N and S. The heterocycloalkyl group may be attached at any heteroatom or carbon atom of the ring such that the result is a stable structure.

Examples of suitable heteroaryl groups include, but are not limited to, pyrrolinyl, pyrrolidinyl, dioxalanyl, imidazolinyl, imidazolidinyl, pyrazolinyl, pyrazolidinyl, piperidinyl, dioxanyl, morpholinyl, dithianyl, thiomorpholinyl, piperazinyl, trithianyl, indolinyl, chromenyl, 3,4-methylenedioxyphenyl, 2,3-dihydrobenzofuryl, dihydrofuryl, and the like. Preferred heterocycloalkyl groups include dihydrofuryl, morpholinyl, piperidinyl, and pyrrolidinyl.

With reference to substituents, the term “independently” means that when more than one of such substituents is possible, such substituents may be the same or different from each other.

Abbreviations used in the specification, particularly the Schemes and Examples, are as follows:

| | |
|---|---|
| DCM = | Dichloromethane |
| DIBAL-H = | Diisobutyl aluminum hydride |
| DIPEA = | Di-isopropylethylamine |
| DMAC = | N,N-Dimethylacetamide |
| DMEM = | Dulbecco's Modified Eagle Medium (Gibco) |
| DMF = | N,N-Dimethylformamide |
| DMSO = | Dimethylsulfoxide |
| DTT = | Dithiothreitol |
| Et₂O = | Diethyl Ether |
| EtOH = | Ethanol |
| HEPES = | 4-(2-Hydroxyethyl)-1-Piperizine Ethane Sulfonic Acid |
| HPLC = | High Pressure Liquid Chromatography |
| LAH = | Lithium aluminum hydride |
| MeOH = | Methanol |
| MOM-Cl = | Methoxymethyl chloride |
| MsCl = | Methanesulfonyl chloride |
| NBuLi = | n-Butyl lithium |
| NMP = | N-Methyl pyrrolodinone |
| NMR = | Nuclear Magnetic Resonance |
| PBS = | Phosphate Buffered Saline |
| TBS = | t-Butyldimethylsilane |
| TBSCl = | t-Butyldimethylchlorosilane |
| TEA = | Triethylamine |
| THF = | Tetrahydrofuran |
| TLC = | Thin Layer Chromatography |

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. Wherein the present invention directed to co-therapy comprising administration of one or more compound(s) of formula (I), compound(s) of formula (II) and/or compound(s) of formula (III) and a progestogen or progestogen antagonist, “therapeutically effective amount” shall mean that amount of the combination of agents taken together so that the combined effect elicits the desired biological or medicinal response. For example, the therapeutically effective amount of co-therapy comprising administration of a compound of formula (I) and progestogen would be the amount of the compound of formula (I) and the amount of the progestogen that when taken together or sequentially have a combined effect that is therapeutically effective. Further, it will be recognized by one skilled in the art that in the case of co-therapy with a therapeutically effective amount, as in the example above, the amount of the compound of formula (I) and/or the amount of the progestogen or progestogen antagonist individually may or may not be therapeutically effective.

As used herein, the term “co-therapy” shall mean treatment of a subject in need thereof by administering one or more compounds of formula (I) with a progestogen or progestogen antagonist, wherein the compound(s) of formula (I) and the progestogen or progestogen antagonist are administered by any suitable means, simultaneously, sequentially, separately or in a single pharmaceutical formulation. Where the compound(s) of formula (I) and the progestogen or progestogen antagonist are administered in separate dosage forms, the number of dosages administered per day for each compound may be the same or different. The compound(s) of formula (I) and the progestogen or progestogen antagonist may be administered via the same or different routes of administration. Examples of suitable methods of administration include, but are not limited to, oral, intravenous (iv), intramuscular (im), subcutaneous (sc), transdermal, and rectal. Compounds may also be administered directly to the nervous system including, but not limited to, intracerebral, intraventricular, intracerebroventricular, intrathecal, intracisternal, intraspinal and/or peri-spinal routes of administration by delivery via intracranial or intravertebral needles and/or catheters with or without pump devices. The compound(s) of formula I and the progestogen or progestogen antagonist may be administered according to simultaneous or alternating regimens, at the same or different times during the course of the therapy, concurrently in divided or single forms.

As used herein, the term “composition” is intended to encompass a product comprising the specified ingredients in the specified amounts, as well as any product which results, directly or indirectly, from combinations of the specified ingredients in the specified amounts.

As used herein, the term “disease or disorder modulated by an estrogen receptor” shall mean any disease or disorder which is mediated by the estrogen α, any disease or disorder which is mediated by the estrogen β receptor or any disease or disorder which is mediated by both the estrogen α and estrogen β receptors. For example, hot flashes, vaginal dryness, osteopenia, osteoporosis, hyperlipidemia, loss of cognitive function, a degenerative brain disorder, cardiovascular disease, cerebrovascular disease breast cancer, endometrial cancer, cervical cancer, prostate cancer, benign prostatic hyperplasia (BPH), endometriosis, uterine fibroids, osteoarthritis and contraception.

As used herein, the term “degenerative brain disease” shall include cognitive disorder, dementia, regardless of underlying cause and Alzheimers disease.

As used herein, the term “cardiovascular disease” shall include elevated blood lipid levels, coronary arthrosclerosis and coronary heart disease.

As used herein, the term “cerebrovascular disease” shall include abnormal regional cerebral blood flow and ischemic brain damage.

Compounds of formula (I) may be prepared according to the process outlined in Scheme 1.

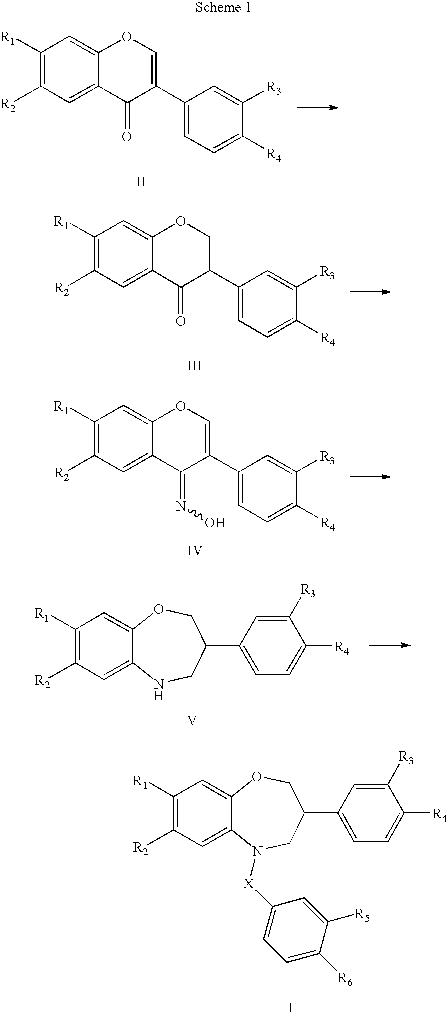

Accordingly, a suitably substituted compound of formula (II), a known compound or a compound prepared by known methods, is reacted with a suitable reducing agent such as DiBAI-H and the like in an organic solvent such as dichloromethane, THF, toluene and the like at reduced temperature, preferably −78° C. to afford the corresponding compound of formula (III).

The compound of formula (III) is reacted with hydroxylamine hydrochloride in a suitable base-solvent mixture such as pyridine-ethanol and the like at elevated temperature to yield the compound of formula (IVI).

The compound of formula (IV) is reacted with lithium hydride in a suitable solvent such as ether, THF, dioxane and the like at reflux temperature to yield the compound of formula (V). Optionally, a compound of formula (V) wherein one of either R1 and R2 and one of either R3 and R4 is methoxy can be reacted with pyridine hydrochloride at elevated temperature, preferably 200° C. to afford the compound wherein the methoxy groups have been converted to the corresponding phenols. This product can be further reacted with a silyl chloride such as tert-Butyldimethyl silyl chloride and the like in the presence of a suitable base such as triethylamine and the like in a suitable organic solvent such as THF, DMF, DMAC and the like to provide the bis TBS-protected phenol.

The compound of formula (V) is reacted with a suitable electrophile such as an acid chloride (for X═CO), a benzyl bromide (for X═CH₂) of a sulfonyl chloride (for X═SO₂) in an appropriate organic solvent such as dichloromethane, THF, pyridine and the like in the presence of a suitable base such as triethylamine and the like to yield the compound of formula (I). Optionally, a compound of formula (I) wherein one of either R1 and R2 and one of either R3 and R4 is methoxymethyleneoxy or tert-butylsilyloxy can be reacted with comncentrated hydrochloric acid in and appropriate organic solvent mixture such as THF-isopropanol and the like to afford the free bis-phenol.

The following Examples are set forth to aid in the understanding of the invention, and are not intended and should not be construed to limit in any way the invention set forth in the claims which follow thereafter.

### EXAMPLE 1

### 7-Methoxy-3-(4-methoxy-phenyl)-chromen-4-one

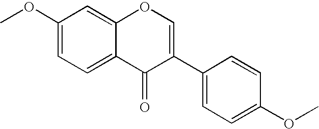

Formononetin (25.2 g, 93.9 mmol) was dissolved in a mixture of anhydrous DMF (125 mL) and acetone (125 mL). Anhydrous potassium carbonate (13.0 g, 93.9 mmol) and dimethyl sulfate (8.90 mL, 93.9 mmol) were added sequentially and the mixture heated to 70° C. for 4 h. After cooling the reaction mixture was poured onto water (1 L). The resultant yellow precipitate was collected by filtration and dried in vacuo to afford the title compound (25.5 g, 96%).

MS (m/Z)=283 (MH+)

### EXAMPLE 2

### 7-Methoxy-3-(4-methoxy-phenyl)-chroman-4-one

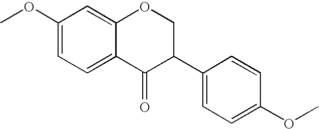

7-Methoxy-3-(4-methoxy-phenyl)-chromen-4-one (13.0 g, 46.0 mmol) was dissolved in dry THF (400 mL) and cooled to −78° C. DIBAL-H (1.5 M, 79 mL, 0.18 mol) was added dropwise over 20 min, and the reaction was stirred at −78° C. for 1 h. The reaction mixture was quenched with Rochelle's solution (150 mL) and stirred at 22° C. for 24 h. The reaction mixture was extracted with CH₂Cl₂ (200 mL×2). The organic layer was washed with H₂O (500 mL×3), dried over Na₂SO₄, and condensed in vacuo to afford the title compound as a yellow semi solid in quantitative yield.

MS (m/Z)=285 (MH+)

### EXAMPLE 3

### 7-Methoxy-3-(4-methoxy-phenyl)-chroman-4-one oxime

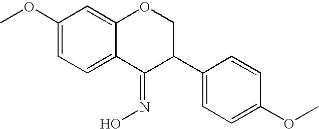

A mixture of 7-Methoxy-3-(4-methoxy-phenyl)-chroman-4-one (13.14 g, 46.22 mmol) and hydroxylamine hydrochloride (12.85 g, 184 mmol) dissolved in a 1:1 mixture of pyridine and ethanol (20 mL) was heated to reflux under argon for 1 hour. The mixture was poured onto water (100 mL) and the precipitate collected by vacuum filtration to afford the oxime (11.9 g, 86%) as a white solid.

MS (m/Z)=300 (MH+)

### EXAMPLE 4

### 3-Methoxy-7-(4-methoxy-phenyl)-6,7,8,9-tetrahydro-5-oxa-9-aza-benzocycloheptene

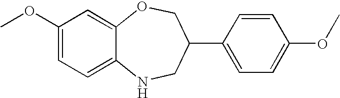

A solution of LAH (53.0 mL, 1.0 M, 53.0 mmol) in ether under argon was treatd with a solution of 7-Methoxy-3-(4-methoxy-phenyl)-chroman-4-one oxime (2.0 mg, 6.7 mmol) in THF (15 mL). The reaction mixture was heated to reflux for 6 hours and then quenched at −5° C. by the cautious addition of Rochelle's solution (45 mL). After stirring overnight at ambient temperature, the mixture was extracted with ether. The organic layers were concentrated and the residue purified by flash chromatography (5% ether/chloroform) to afford 1.2 g (63%) of the product as a colorless gum.

MS (m/Z)=286 (MH+)

### EXAMPLE 5

### 7-Methoxy-3-(3-methoxy-phenyl)-chromen-4-one

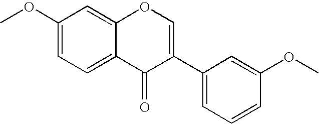

7-Hydroxy-3-(3-methoxy-phenyl)-chromen-4-one (26.4 g, 98.3 mmol) was dissolved in a mixture of anhydrous DMF (125 mL) and acetone (100 mL). Anhydrous potassium carbonate (13.6 g, 98.3 mmol) and dimethyl sulfate (9.5 mL, 100 mmol) were added sequentially and the mixture heated to 70° C. for 4 h. After cooling the reaction mixture was poured onto water (1 L). The resultant yellow precipitate was collected by filtration and dried in vacuo to afford the title compound (25.4 g, 91%).

MS (m/Z)=283 (MH+)

### EXAMPLE 6

### 7-Methoxy-3-(3-methoxy-phenyl)-chroman-4-one

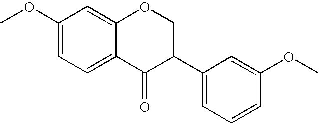

7-Methoxy-3-(3-methoxy-phenyl)-chromen-4-one (13.0 g, 46.0 mmol) was dissolved in dry THF (400 mL) and cooled to −78° C. DIBAL-H (1.5 M, 79 mL, 0.18 mol) was added dropwise over 20 min, and the reaction was stirred at −78° C. for 1 h. The reaction mixture was quenched with Rochelle's solution (150 mL) and stirred at 22° C. for 24 h. The reaction mixture was extracted with CH₂Cl₂ (200 mL×2). The organic layer was washed with H₂O (500 mL×3), dried over Na₂SO₄, and condensed in vacuo to afford the title compound as a yellow semi solid in quantitative yield.

MS (m/Z) 285 (MH+)

### EXAMPLE 7

### 7-Methoxy-3-(3-methoxy-phenyl)-chroman-4-one oxime

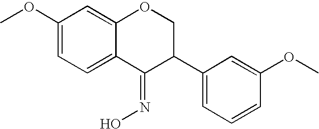

A mixture of 7-Methoxy-3-(4-methoxy-phenyl)-chroman-4-one (8.31 g, 29.2 mmol) and hydroxylamine hydrochloride (8.12 g, 117 mmol) dissolved in a 1:2 mixture of pyridine and ethanol (75 mL) was heated to reflux under argon for 1 hour. The mixture was poured onto water (100 mL) and the precipitate collected by vacuum filtration to afford the oxime (3.64 g, 42%) as a white solid.

MS (m/Z)=300 (MH+)

### EXAMPLE 8

### 3-Methoxy-7-(3-methoxy-phenyl)-6,7,8,9-tetrahydro-5-oxa-9-aza-benzocycloheptene

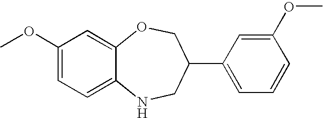

A solution of LAH (100 mL, 1.0 M, 100 mmol) in ether under argon was treated with a solution of 7-Methoxy-3-(4-methoxy-phenyl)-chroman-4-one oxime (2.0 mg, 6.7 mmol) in THF (15 mL). The reaction mixture was heated to reflux for 6 hours and then quenched at −5° C. by the cautious addition of Rochelle's solution (100 mL). After stirring overnight at ambient temperature, the mixture was extracted with ether. The organic layers were concentrated and the residue purified by flash chromatography (5% ether/chloroform) to afford 1.34 g (39%) of the product as a yellow oil.

MS (m/Z)=286 (MH+)

### EXAMPLE 9

### 7-(3-Hydroxy-phenyl)-6,7,8,9-tetrahydro-5-oxa-9-aza-benzocyclohepten-3-ol

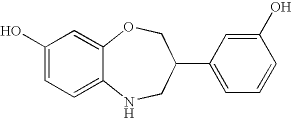

A solution of 3-Methoxy-7-(3-methoxy-phenyl)-6,7,8,9-tetrahydro-5-oxa-9-aza-benzocycloheptene (1.3 g, 4.56 mmol, prepared from 7-Methoxy-3-(3-methoxy-phenyl)-chroman-4-one oxime as described in example 24) and pyridine hydrochloride (5.3 g, 45.6 mmol) was heated to 200° C. under nitrogen for 3 h. The residue was dissolved in MeOH and treated with NaOMe (8.2 mmol, 25% in MeOH, 36 mmol) and concentrated to dryness. The residue was dissolved in THF/MeOH (5:1) and the white solid removed by filtration (three times) to afford, after concentration in vacuo the title compound as a brown oil in quantitative yield.

MS (m/Z)=256 (M−H)

### EXAMPLE 10

### 3-(tert-Butyl-dimethyl-silanyloxy)-7-[3-(tert-butyl-dimethyl-silanyloxy)-phenyl]-6,7,8,9-tetrahydro-5-oxa-9-aza-benzocycloheptene

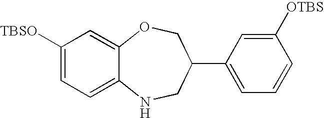

The crude product from example 9 was dissolved in DMF (13 mL) and treated with TBSCI (1.5 g, 10.0 mmol) and imidazole (1.86 g, 27.4 mmol) and stirred at room temperature for 2 h. The mixture was poured onto water and extracted with ether. The combined extracts were concentrated in vacuo and the residue purified by flash chromatography (5-10% ether/pentane) to afford the product as a brown oil (1.35 g, 61% form the bis-methoxy compound).

MS (m/Z)=486 (MH+)

### EXAMPLE 11

### 7-(4-Hydroxy-phenyl)-6,7,8,9-tetrahydro-5-oxa-9-aza-benzocyclohepten-3-ol

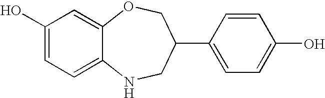

A solution of 3-Methoxy-7-(4-methoxy-phenyl)-6,7,8,9-tetrahydro-5-oxa-9-aza-benzocycloheptene (1.0 g, 3.5 mmol) and pyridine hydrochloride (6.0 g, 52 mmol) was heated to 200° C. under nitrogen for 3 h. The residue was dissolved in MeOH and treated with NaOMe (52 mmol) and concentrated to dryness. The residue was dissolved in THF/MeOH (5:1) and the white solid removed by filtration (three times) to afford, after concentration in vacuo the title compound as a brown oil in quantitative yield.

MS (m/Z)=256 (M−H)

### EXAMPLE 12

### 3-(tert-Butyl-dimethyl-silanyloxy)-7-[4-(tert-butyl-dimethyl-silanyloxy)-phenyl]-6,7,8,9-tetrahydro-5-oxa-9-aza-benzocycloheptene

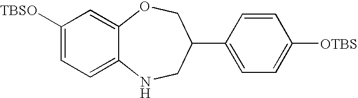

The crude product from example 11 was dissolved in DMF (20 mL) and treated with TBSCI (1.58 g, 10.5 mmol) and imidazole (768 mg, 11.3 mmol) and stirred at room temperature for 12 h. The mixture was poured onto water and extracted with ether. The combined extracts were concentrated in vacuo and the residue purified by flash chromatography (DCM) to afford the product (0.73 g, 43% from the bis-methoxy compound).

MS (m/Z)=486 (MH+)

### EXAMPLE 13

### 3-(tert-Butyl-dimethyl-silanyloxy)-7-[3-(tert-butyl-dimethyl-silanyloxy)-phenyl]-9-[4-(2-chloro-ethoxy)-benzyl]-6,7,8,9-tetrahydro-5-oxa-9-aza-benzocycloheptene

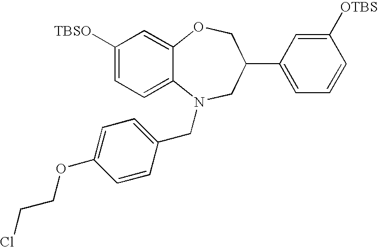

A mixture of the material from example 10 (650 mg, 1.34 mmol), 1-Bromomethyl-4-(2-chloro-ethoxy)-benzene (468 mg, 1.88 mmol) and anhydrous potassium carbonate (259 mg, 1.88 mmol) was dissolved in dry DMF (12 mL) and stirred for 2 h at 70° C. The reaction mixture was poured onto water and extracted with ether. The organic extracts were concentrated and the residue purified via flash chromatography to produce 290 mg (32%) of the title compound.

MS (m/Z) 654 (MH+)

### EXAMPLE 14

### 3-(tert-Butyl-dimethyl-silanyloxy)-7-[3-(tert-butyl-dimethyl-silanyloxy)-phenyl]-9-[4-(2-piperidin-1-yl-ethoxy)-benzyl]-6,7,8,9-tetrahydro-5-oxa-9-aza-benzocycloheptene

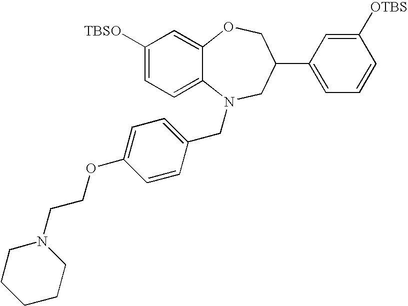

This material from Example 13 was dissolved in dry DMF (10 mL) and treated with potassium iodide (80 mg, 0.49 mmol) and piperidine (1.1 mL, 11 mmol). The mixture was heated at 60° C. overnight, cooled, poured onto sat. sodium bicarbonate soluton and extracted with ether. The combined extracts were concentrated and the residue purified by flash chromatography (70% ether/pentane) to afford the title compound as a pasty solid (83 mg, 27%).

MS (m/Z)=703 (MH+)

### EXAMPLE 15

### 7-(3-Hydroxy-phenyl)-9-[4-(2-piperidin-1-yl-ethoxy)-benzyl]-6,7,8,9-tetrahydro-5-oxa-9-aza-benzocyclohepten-3-ol

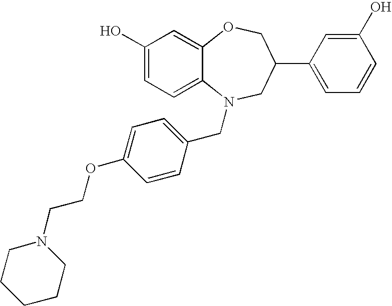

The material from Example 14 (83 mg, 0.12 mmol) was dissolved in a mixture of MeOH and THF (1:1, 2 mL), treated with HCl (1N, 0.5 mL, 0.5 mmol) and stirred for 2 h at 50° C. The reaction mixture was concentrated in vacuo, the residue washed with pentane/ether (1:1, 2×50 mL) and dried in vacuo to produce the title compound (32 mg, 57%).

MS (m/Z)=475 (MH+)

### EXAMPLE 16

### 3-(tert-Butyl-dimethyl-silanyloxy)-7-[4-(tert-butyl-dimethyl-silanyloxy)-phenyl]-9-[4-(2-chloro-ethoxy)-benzyl]-6,7,8,9-tetrahydro-5-oxa-9-aza-benzocycloheptene

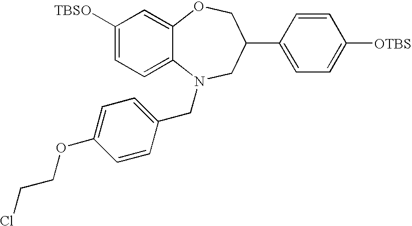

A mixture of material from example 12 (287 mg, 0.59 mmol), 1-Bromomethyl-4-(2-chloro-ethoxy)-benzene (176 mg, 0.71 mmol) and anhydrous potassium carbonate (96 mg, 0.69 mmol) was dissolved in dry DMF (12 mL) and stirred for 2 h at 70° C. The reaction mixture was poured onto water and extracted with ether. The organic extracts were concentrated and the residue purified via flash chromatography to produce 190 mg (49%) of the title compound.

MS (m/Z)=654 (MH+)

### EXAMPLE 17

### 9-[4-(2-Chloro-ethoxy)-benzyl]-7-(4-hydroxy-phenyl)-6,7,8,9-tetrahydro-5-oxa-9-aza-benzocyclohepten-3-ol

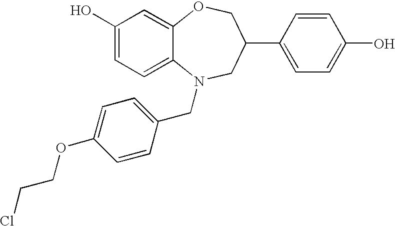

A mixture of material from example 16 (37 mg, 0.057 mmol) was dissolved in a mixture of MeOH and THF (1:1, 10 mL), treated with HCl (1N, 1.0 mL, 1.0 mmol) and stirred for 2 h at 55° C. The reaction mixture was concentrated in vacuo, the residue washed with pentane/ether (1:1, 2×50 mL) and dried in vacuo to produce 19 mg (78%) of the title compound as a brown solid.

MS (m/Z)=426 (MH+)

### EXAMPLE 18

### 7-(4-Hydroxy-phenyl)-9-[4-(2-piperidin-1-yl-ethoxy)-benzyl]-6,7,8,9-tetrahydro-5-oxa-9-aza-benzocyclohepten-3-ol

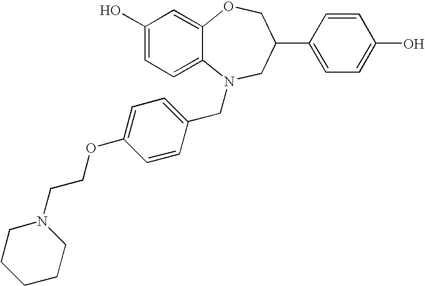

This material was prepared in two steps from Example 16 by a displacement of the chloride by piperidine (as described in example 14) followed by acidic hydrolysis of the TBS groups (as described in example 15).

MS (m/Z)=475 (MH+)

### EXAMPLE 19

### [4-(2-Chloro-ethoxy)-phenyl]-[3-methoxy-7-(4-methoxy-phenyl)-7,8-dihydro-6H-5-oxa-9-aza-benzocyclohepten-9-yl]-methanone

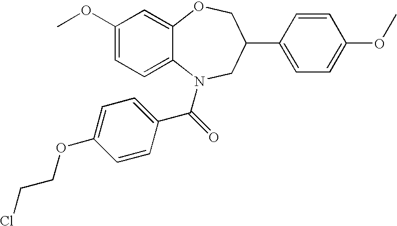

A solution of the material from example 4 (87 mg, 0.30 mmol), 4-(2-Chloro-ethoxy)-benzoyl chloride (110 mg, 0.50 mmol) and triethylamine (0.04 mL, 0.30 mmol) in dry THF (4 mL) was stirred overnight at ambient temperature. Water was added and the mixture extracted with ethyl acetate. The organic layers were concentrated and the residue purified by prep tlc (1:1 ether/pentane) to afford 36 mg of the title compound.

MS (m/Z)=468 (MH+)

### EXAMPLE 20

### [3-Methoxy-7-(4-methoxy-phenyl)-7,8-dihydro-6H-5-oxa-9-aza-benzocyclohepten-9-yl]-[4-(2-piperidin-1-yl-ethoxy)-phenyl]-methanone

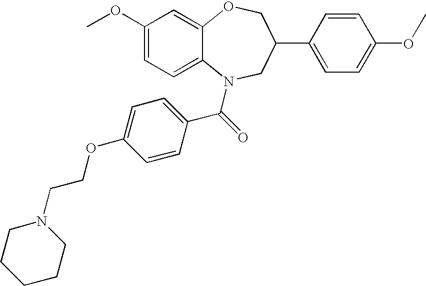

This material from example 19 (36 mg, 0.077 mmol) was dissolved in dry DMF (2 mL) and treated with piperidine (0.19 mL, 1.9 mmol) and potassium iodide (14 mg, 0.085 mmol). The resulting mixture was heated to 50° C. overnight, cooled and extracted with ethyl acetate. After concentration in vacuo, the residue was purified by prep tlc (5% MeOH/DCM) to produce the title compound (35 mg, 88%).

MS (m/Z)=517 (MH+)

### EXAMPLE 21

### 3-(tert-Butyl-dimethyl-silanyloxy)-7-[4-(tert-butyl-dimethyl-silanyloxy)-phenyl]-9-[3-(2-chloro-ethoxy)-benzyl]-6,7,8,9-tetrahydro-5-oxa-9-aza-benzocycloheptene

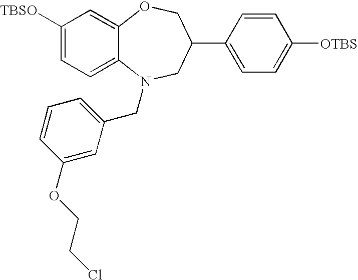

A mixture of material from example 12 (488 mg, 1.01 mmol), 1-Bromomethyl-3-(2-chloro-ethoxy)-benzene (296 mg, 1.19 mmol) and treithylamine (0.30 mL, 2.15 mmol) was dissolved in dry DMF (3 mL) and stirred for 0.5 h at 70° C. and overnight at room temperature. The reaction mixture was poured onto water and extracted with ether. The organic extracts were concentrated and the residue purified via flash chromatography to produce the title compound.

MS (m/Z)=654 (MH+)

### EXAMPLE 22

### 9-[3-(2-Chloro-ethoxy)-benzyl]-7-(4-hydroxy-phenyl)-6,7,8,9-tetrahydro-5-oxa-9-aza-benzocyclohepten-3-ol

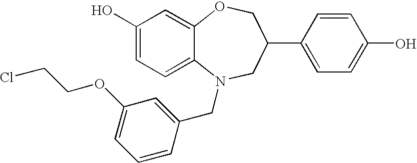

A mixture of material from example 21 (955 mg, 1.46 mmol) was treated with TBAF (2.9 mL, 1.0 M in THF, 2.9 mmol) and stirred overnight at room temperature. The reaction mixture was concentrated in vacuo and the residue purified by flash chromatography to afford the title compound (460 mg, 74%).

MS (m/Z)=424 (M−H)

### EXAMPLE 23

### 9-[3-(2-Chloro-ethoxy)-benzyl]-3-methoxymethoxy-7-(4-methoxymethoxy-phenyl)-6,7,8,9-tetrahydro-5-oxa-9-aza-benzocycloheptene

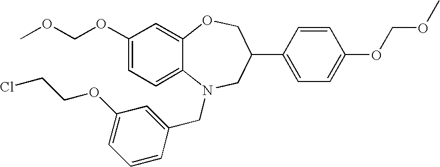

A mixture of material from example 22 (460 mg, 1.08 mmol) was dissolved in dry THF (10 mL), treated with sodium hydride (97 mg, 60% in mineral oil, 2.43 mmol), chloromethylmethyl ether (0.19 mL, tech grade, ˜2.5 mmol) and stirred overnight at room temperature. The reaction mixture was poured onto water and extracted with DCM. The organic extracts were concentrated in vacuo and the residue purified by flash chromatography (0-10% MeOH/DCM) to afford the title compound (460 mg, 74%).

MS (m/Z)=514 (MH+)

### EXAMPLE 24

### 3-Methoxymethoxy-7-(4-methoxymethoxy-phenyl)-9-[3-(2-piperidin-1-yl-ethoxy)-benzyl]-6,7,8,9-tetrahydro-5-oxa-9-aza-benzocycloheptene

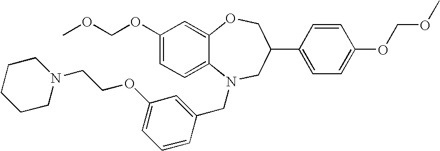

Using the procedure described in Example 20 on the material prepared in example 23 (0.23 mmol scale), the title compound was prepared.

MS (m/Z)=563 (MH+)

### EXAMPLE 25

### 7-(4-Hydroxy-phenyl)-9-[3-(2-piperidin-1-yl-ethoxy)-benzyl]-6,7,8,9-tetrahydro-5-oxa-9-aza-benzocyclohepten-3-ol

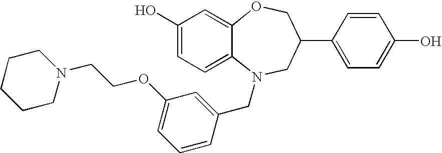

The material from example 24 was treated with a mixture of isopropanol, THF and concentrated HCl at room temperature to deliver the title compound in 66% overall yield (over 2 steps from the material produced in example 23).

MS (m/Z)=475 (MH+)

### EXAMPLE 26

### {3-(tert-Butyl-dimethyl-silanyloxy)-7-[3-(tert-butyl-dimethyl-silanyloxy)-phenyl]-7,8-dihydro-6H-5-oxa-9-aza-benzocyclohepten-9-yl}-[3-(2-chloro-ethoxy)-phenyl]-methanone

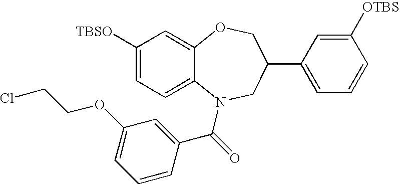

A solution of the material from example 10 (216 mg, 0.45 mmol), 3-(2-Chloro-ethoxy)-benzoyl chloride (0.96 mmol) and triethylamine (1.00 mL, 7.2 mmol) in dry 1,2-dichloroethane (4 mL) was stirred 0.33 h at 45° C. The reaction mixture was concentrated and the residue purified by flash chromatography to afford the title compound.

MS (m/Z)=668 (MH+)

### EXAMPLE 27

### {3-(tert-Butyl-dimethyl-silanyloxy)-7-[3-(tert-butyl-dimethyl-silanyloxy)-phenyl]-7,8-dihydro-6H-5-oxa-9-aza-benzocyclohepten-9-yl}-[3-(2-piperidin-1-yl-ethoxy)-phenyl]-methanone

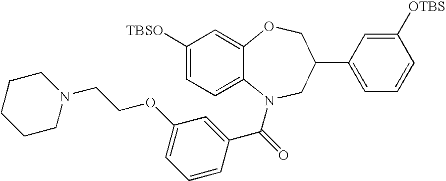

This material from example 26 (177 mg, 0.26 mmol) was dissolved in dry DMF (2 mL) and treated with piperidine (0.50 mL, 5.1 mmol) and potassium iodide (48 mg, 0.29 mmol). The resulting mixture was heated to 50° C. overnight, cooled and extracted with ethyl acetate. After concentration in vacuo, the residue was purified by prep tlc (5% MeOH/DCM) to produce the title compound.

MS (m/Z)=717 (MH+)

### EXAMPLE 28

### [3-Hydroxy-7-(3-hydroxy-phenyl)-7,8-dihydro-6H-5-oxa-9-aza-benzocyclohepten-9-yl]-[3-(2-piperidin-1-yl-ethoxy)-phenyl]-methanone

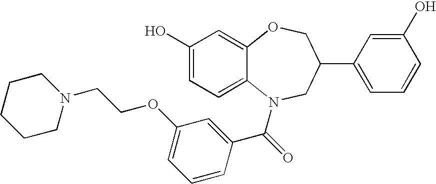

This material from example 27 was deprotected as described in example 17 to produce the title compound.

MS (m/Z)=489 (MH+)

### EXAMPLE 29

### Estrogen Receptor α Flash Plate Assay

This assay monitors binding of radio-labeled estrogen to the estrogen receptor. It is performed on a BioMek 2000 (Beckman). Plates are read in a scintillation counter (Packard TopCount), with decreased counts an indication of binding of a compound to the receptor. The assay was run according to the procedure described by Allan, et al., Anal. Biochem. (1999), 275(2), 243-247.

On day one, 100 μL of Estrogen Screening Buffer (ESB, Panvera) containing 5 mM dithiothreitol (DTT, Panvera), 0.5 μg mouse anti-estrogen receptor monoclonal antibody (SRA-1010, Stressgen) and 50 ng purified human estrogen receptor α (Panvera) were added to each well of a 96 well FlashPlate Plus plate crosslinked with goat anti-mouse antibodies (NEN Life Sciences). The plate was sealed and incubated at 4° C. overnight.

On day two, each well was washed three times with 200 μL PBS, pH 7.2, at room temperature. To each well was then added 98 μL radio-labeled estrogen (0.5 nM, which equals 6 nCi for a 120 Ci/mmol batch, Amersham), diluted in ESB and 5 mM dithiothreitol (DTT). To individual wells were then added 2.5 μL test compound diluted in 30% (v/v) dimethyl sulfoxide/50 mM HEPES, pH 7.5. The wells were mixed three times by aspiration, the plate sealed and incubated at room temperature for one hour. The wells were then counted for 1 min in a TopCount scintillation counter (Packard).

### EXAMPLE 30

### Estrogen Receptor β Fluorescence Polarization Assay

This assay monitors binding of a fluorescent analog of estrogen (Fluormone ES2, Panvera) to the estrogen receptor. Plates are read in a fluorometer that can be set to polarization mode. A decrease in fluorescence relative to vehicle control is an indication of binding of a compound to the receptor.

It is crucial to avoid introduction of air bubbles into the reaction in each well of the 96 well plate throughout this procedure. (Bubbles on the surface of the reaction disrupt light flow, affecting the polarization reading.) However, it is also crucial to effectively mix the reaction components upon addition to the well.

On ice, a 2× standard mixture of Assay Buffer (Panvera), 10 nM DTT and 40 nM ES2 was prepared. On ice, a 2× reaction mixture of Assay Buffer (Panvera), and 20 nM hER-β (Panvera) and 40 nM ES2 was also prepared.

Dilutions of test compound were prepared in 30% (v/v) dimethyl sulfoxide/50 mM HEPES, pH 7.5. At this point, the dilutions were 40× the final required concentration.

The standard mixture at 50 μL was then added to each well. The reaction mixture at 48 μL was added to all wells. The compound dilution at 2.5 μL was added to the appropriate wells. The reaction mixtures were mixed using a manual pipette, a roll of aluminum foil adhesive cover was placed on the plate and the plate incubated at room temperature for 1 hour.

Each well on the plate was then read in an LjL Analyst with an excitation wavelength of 265 nm and an emission wavelength of 538.

Representative compound of the present invention were tested according to the procedure described above for binding to the Estrogen Receptor a and Estrogen Receptor β, with results as listed in Table 6.

TABLE 6

| | ERα Binding | ERβ Binding |
|---|---|---|
| ID # | IC₅₀ (μM) | IC₅₀ (μM) |
| 8 | 2.9 | >100 |
| 9 | 0.53 | 26 |
| 12 | 0.43 | 1.1 |
| 13 | 0.081 | 2.6 |
| 14 | 0.024 | 1.1 |
| 15 | 0.10 | 2.6 |
| 16 | 0.12 | 0.67 |
| 17 | 2.9 | 1.2 |
| 18 | 0.066 | 0.088 |
| 19 | >1 | >1 |
| 20 | >1 | >1 |
| 21 | >1 | >1 |
| 22 | 0.052 | 0.56 |
| 23 | 0.35 | 0.87 |
| 24 | 0.056 | 0.092 |
| 25 | 0.022 | 0.30 |
| 26 | >1 | >1 |

### EXAMPLE 31

### MCF-7 Cell Proliferation Assay

This assay was run according to the procedure described by Welshons, et al., (Breast Cancer Res. Treat., 1987, 10(2), 169-75), with minor modification.

Briefly, MCF-7 cells (from Dr. C. Jordan, Northwestern University) were maintained in RPMI 1640 phenol red free medium (Gibco) in 10% FBS (Hyclone), supplemented with bovine insulin and non-essential amino acid (Sigma). The cells were initially treated with 4-hydroxytamoxifen (10⁻⁸ M) and let stand at 37° C. for 24 hours. Following this incubation with tamoxifen, the cells were treated with compounds at various concentrations.

Compounds to be tested in the agonist mode were added to the culture media at varying concentrations. Compounds to be treated in the antagonist mode were prepared similarly, and 10 nM 17β-estradiol was also added to the culture media. The cells were incubated for 24 hours at 37° C. Following this incubation, 0.1 μCi of ¹⁴C-thymidine (56 mCi/mmol, Amersham) was added to the culture media and the cells were incubated for an additional 24 hours at 37° C. The cells were then washed twice with Hank's buffered salt solution (HBSS) (Gibco) and counted with a scintillation counter. The increase in the ¹⁴C-thymidine in the compound treated cells relative to the vehicle control cells were reported as percent increase in cell proliferation.

### EXAMPLE 32

As a specific embodiment of an oral composition, 100 mg of the Compound #25, is formulated with sufficient finely divided lactose to provide a total amount of 580 to 590 mg to fill a size O hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. A compound of the formula (I) 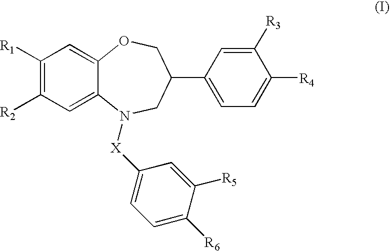
wherein:
R₁, R₂, R₃, and R₄ are selected form the group consisting of hydrogen, halogen, hydroxy, alkyl, alkoxy, acyloxy, silyloxy and hydroxy substituted lower alkyl, provided that at least one of R₁, R₂, R₃ or R₄ is hydroxy, alkoxy, silyloxy or acyloxy;
X is selected from the group consisting of CH₂, CO or SO₂;
R5 and R6 are each independently selected from the group consisting of hydrogen, halogen, alkoxy, lower alkyl, —O—(CH₂)₂₋₃—Cl, —O—(CH₂)₂₋₃—OH, and —O—(CH₂)₂₋₃—NR^{A}R^{B}, provided that if present only one of R5 or R6 is —O—(CH₂)₂₋₃—NR^{A}R^{B};
wherein, R^{A} and R^{B} are each independently selected from the group consisting of hydrogen and lower alkyl; alternatively R^{A} and R^{B} are taken together with the N atom to which they are bound to form a five to six membered heteroaryl or a five to six membered heterocycloalkyl group; or a pharmaceutically acceptable salt thereof.

2. A compound as in claim 1 wherein
X is CH₂
R₂ is hydrogen
R₁ is selected from the group consisting of hydroxy, alkoxy, methoxymethoxy, acyloxy, and silyloxy
R₃ and R₄ are selected such that one of R₃ and R₄ is hydrogen and the other is selected from the group consisting of hydroxy, alkoxy, silyloxy, methoxymethoxy or acyloxy
R₅ and R₆ are selected form a group consisting of hydrogen, halogen, alkoxy, lower alkyl, or —O—(CH₂)₂₋₃—Cl or —O—(CH₂)₂₋₃—NR^{A}R^{B} provided that if present only one of R₅ or R₆ is —O—(CH₂)₂₋₃—NR^{A}R^{B}
R^{A} and R^{B} are independently selected from the group consisting of hydrogen and methyl or are taken together with the nitrogen atom to which they are bound to form pyrrolodinyl, morpholinyl or piperidinyl.

3. A compound as in claim 1 wherein
X is CH₂
R₂ is hydrogen
R₁ is selected from the group consisting of hydroxy, alkoxy, methoxymethoxy, acyloxy, and silyloxy
R₃ and R₄ are selected such that one of R₃ and R₄ is hydrogen and the other is selected from the group consisting of hydroxy, alkoxy, silyloxy, methoxymethoxy or acyloxy
R₅ and R₆ are selected form a group consisting of, hydrogen, —O—(CH₂)₂—Cl or —O—(CH₂)₂—NR^{A}R^{B} provided that if present only one of R₅ or R₆ is —O—(CH₂)₂—NR^{A}R^{B}
R^{A} and R^{B} are independently selected from the group consisting of hydrogen and methyl or are taken together with the nitrogen atom to which they are bound to form pyrrolodinyl, morpholinyl or piperidinyl.

4. A compound as in claim 1 wherein
X is CH₂
R₂ is hydrogen
R₁ is selected from the group consisting of hydroxy, acyloxy, and silyloxy
R₃ and R₄ are selected such that one of R₃ and R₄ is hydrogen and the other is selected from the group consisting of hydroxy, silyloxy, or acyloxy
R₅ and R₆ are selected form a group consisting of hydrogen, —O—(CH₂)₂—Cl or —O—(CH₂)₂—NR^{A}R^{B} provided that if present only one of R₅ or R₆ is —O—(CH₂)₂—NR^{A}R^{B}
R^{A} and R^{B} are taken together with the nitrogen atom to which they are bound to form pyrrolodinyl, morpholinyl or piperidinyl.

5. A compound as in claim 1 wherein
X is CH₂
R₂ is hydrogen
R₁ is hydroxy
R₃ and R₄ are selected such that one of R₃ and R₄ is hydrogen and the other is hydroxy
R₅ and R₆ are selected such that one is hydrogen and the other is —O—(CH₂)₂—NR^{A}R^{B}
R^{A} and R^{B} are taken together with the nitrogen atom to which they are bound to form morpholinyl or piperidinyl.

6. A compound as in claim 1 wherein
X is CO
R₂ is hydrogen
R₁ is selected from the group consisting of hydroxy, alkoxy, methoxymethoxy, acyloxy, and silyloxy
R₃ and R₄ are selected such that one of R₃ and R₄ is hydrogen and the other is selected from the group consisting of hydroxy, alkoxy, silyloxy, methoxymethoxy or acyloxy
R₅ and R₆ are selected form a group consisting of hydrogen, halogen, alkoxy, lower alkyl, or —O—(CH₂)₂₋₃—Cl or —O—(CH₂)₂₋₃—NR^{A}R^{B} provided that if present only one of R₅ or R₆ is —O—(CH₂)₂₋₃—NR^{A}R^{B}
R^{A} and R^{B} are independently selected from the group consisting of hydrogen and methyl or are taken together with the nitrogen atom to which they are bound to form pyrrolodinyl, morpholinyl or piperidinyl.

7. A compound as in claim 1 wherein
X is CO
R₂ is hydrogen
R₁ is selected from the group consisting of hydroxy, alkoxy, methoxymethoxy, acyloxy, and silyloxy
R₃ and R₄ are selected such that one of R₃ and R₄ is hydrogen and the other is selected from the group consisting of hydroxy, alkoxy, silyloxy, methoxymethoxy or acyloxy
R5 and R6 are selected form a group consisting of hydrogen, —O—(CH₂)₂—Cl or —O—(CH₂)₂—NR^{A}R^{B} provided that if present only one of R₅ or R₆ is —O—(CH₂)₂—NR^{A}R^{B}
R^{A} and R^{B} are independently selected from the group consisting of hydrogen and methyl or are taken together with the nitrogen atom to which they are bound to form pyrrolodinyl, morpholinyl or piperidinyl.

8. A compound as in claim 1 wherein
X is CO
R₂ is hydrogen
R₁ is selected from the group consisting of hydroxy, acyloxy, and silyloxy
R₃ and R₄ are selected such that one of R₃ and R₄ is hydrogen and the other is selected from the group consisting of hydroxy, silyloxy, or acyloxy
R₅ and R₆ are selected form a group consisting of hydrogen, —O—(CH₂)₂—Cl or —O—(CH₂)₂—NR^{A}R^{B} provided that if present only one of R₅ or R₆ is —O—(CH₂)₂—NR^{A}R^{B}
R^{A} and R^{B} are taken together with the nitrogen atom to which they are bound to form pyrrolodinyl, morpholinyl or piperidinyl.

9. A compound as in claim 1 wherein
X is CO
R₂ is hydrogen
R₁ is hydroxy
R₃ and R₄ are selected such that one of R3 and R4 is hydrogen and the other is hydroxy
R₅ and R₆ are selected such that one is hydrogen and the other is —O—(CH₂)₂—NR^{A}R^{B}
R^{A} and R^{B} are taken together with the nitrogen atom to which they are bound to form morpholinyl or piperidinyl.

10. [3-Hydroxy-7-(3-hydroxy-phenyl)-7,8-dihydro-6H-5-oxa-9-aza-benzocyclohepten-9-yl]-[3-(2-piperidin-1-yl-ethoxy)-phenyl]-methanone.

11. 7-(4-Hydroxy-phenyl)-9-[3-(2-piperidin-1-yl-ethoxy)-benzyl]-6,7,8,9-tetrahydro-5-oxa-9-aza-benzocyclohepten-3-ol.

12. A pharmaceutical composition comprising a pharmaceutically acceptable carrier and a compound of claim 1.

13. A pharmaceutical composition made by mixing a compound of claim 1 and a pharmaceutically acceptable carrier.

14. A process for making a pharmaceutical composition comprising mixing a compound of claim 1 and a pharmaceutically acceptable carrier.

15. A method of treating a disorder mediated by an estrogen receptor, in a subject in need thereof comprising administering to the subject a therapeutically effective amount of the compound of claim 1.

16. The method of claim 15, wherein the disorder mediated by an estrogen receptor is selected from the group consisting of hot flashes, vaginal dryness, osteopenia, osteoporosis, hyperlipidemia, loss of cognitive function, degenerative brain diseases, cardiovascular diseases, cerebrovascular diseases, cancer of the breast tissue, hyperplasia of the breast tissue, cancer of the endometrium, hyperplasia of the endometrium, cancer of the cervix, hyperplasia of the cervix, cancer of the prostate, hyperplasia of the prostate, endometriosis, uterine fibroids, osteoarthritis and contraception.

17. The method of claim 15, wherein the disorder mediated by an estrogen receptor is selected from the group consisting of osteoporosis, hot flashes, vaginal dryness, breast cancer and endometriosis.

18. A method of treating a disorder mediated by an estrogen receptor in a subject in need thereof comprising administering to the subject a therapeutically effective amount of the composition of claim 12.

19. A method of contraception comprising co-therapy with a therapeutically effective amount of a compound as in claim 1 and a progestogen or a progestogen antagonist.
